(19) United States
(12) Patent Application Publication
(10) Pub. No.: US 2007/0089748 A1
(43) Pub. Date: Apr. 26, 2007

(54) Tracheal catheter with closeable suction lumen

(76) Inventors: Edward B. Madsen, Riverton, UT (US); Scott M. Teixeira, Draper, UT (US)

Correspondence Address: KIMBERLY-CLARK WORLDWIDE, INC., 401 NORTH LAKE STREET, NEENAH, WI, 54956, US

(21) Appl. No.: 11/259,724

(22) Filed: Oct. 26, 2005

(51) Int. Cl. A62B 9/06; A61M 16/00

(52) U.S. Cl. 128/207.15; 128/207.14

(57) ABSTRACT

A multilumen tracheal tube and a method of suctioning fluids through the same are disclosed. The tube includes a rinse lumen and a suction lumen, the rinse lumen enables rinsing of fluids within the suction lumen while simultaneously suctioning the fluids and ventilating the patient. A closure mechanism is provided to seal off the interior of the tracheal tube during the rinse function. This enables higher rinse pressures and greater suctioning capabilities.

### BACKGROUND

The present invention relates to a tracheal tube used for mechanical ventilation of a hospital patient, by insertion of the tube into the trachea of the patient. In particular, the present invention relates to a tracheal tube having means for irrigating and/or evacuating contaminated secretions accumulating above the tracheal tube cuff and thereby reducing the risk of such contaminated secretions entering the lungs of the patient.

Endotracheal intubation involves the insertion of a tubular device, known as an endotracheal tube, into the trachea of a patient. The endotracheal tube passes through the trachea and terminates at a position above the carina, anterior to a position between the second and fourth thoracic vertebrate. Gases may then be introduced through the endotracheal tube and into the lungs of the patient.

The primary purposes of endotracheal intubation, are to mechanically ventilate the patient's lungs, when a disease prevents the patient from normal, breathing induced ventilation, or to apply anesthetic gases during surgical intervention. In order to create enough air pressure to accomplish such mechanical ventilation and to prevent escape of gases past the tube, it is necessary to seal the passageway around the endotracheal tube. A seal may be produced by the use of an inflatable cuff formed integrally with and surrounding the endotracheal tube. When the endotracheal tube has been introduced into the patient's trachea, the inflatable cuff will normally be located about 3 to 5 centimeters above the carina and within the tube-like trachea.

The inflatable cuff is then inflated so as to engage the wall of the trachea and thereby seal the trachea and prevent gases being introduced through the tracheal tube from simply backing up around the tube. While treatment of this sort has proved successful for patients having chronic or acute respiratory diseases, there is a constant risk of several complications.

In particular, many patients receiving endotracheal intubation develop pneumonia, resulting from an infection of the lungs, possibly induced by contaminated, pooled secretions entering the trachea and the lungs after bypassing the epiglottis during intubation. The epiglottis normally operates as a valve which selectively closes the entry into the trachea and lungs, to prevent the introduction of secretions and particulate matter. However, when a tracheal tube is in place, the epiglottis is held in an open position, and secretions which would normally be directed away from the trachea and into the digestive system, instead follow the path of the endotracheal tube and pool above the inflatable cuff of the endotracheal tube.

The greatest risk of such infectious secretions reaching the lungs is upon the cessation of mechanical ventilation. In particular, when the need for endotracheal intubation ends, the inflatable cuff of the endotracheal tube is deflated so that the endotracheal tube may be withdrawn from the patient. The infectious secretions which have pooled above the inflatable cuff are then released and are free to flow into the lungs, where bronchitis or pneumonia may rapidly develop. There is also the risk of the infectious secretions reaching the lungs during the intubation, by aspiration of the secretions past the tracheal tube cuff.

To overcome these risks, it is known in the prior art to combine a single lumen suction tube with a tracheal tube. The suction tube is joined to the endotracheal tube in a suitable manner, the end of the suction tube terminating at a position above the inflatable cuff. The suction tube provides means for suction or evacuation of any pooled secretions which accumulate in the trachea above the inflatable cuff. However, such prior art devices have the disadvantage that use of a single lumen for the suction tube often causes direct suction to be exerted on the tracheal mucosa which may then result in damage to the mucosa.

U.S. Pat. No. 4,840,173 to Porter III, describes an endotracheal tube having a single lumen suction tube merged thereto. In particular, this patent describes a device wherein the suction tube is laminated to the outside of the ventilation tube, so that the suction tube terminates at a position just above the inflatable cuff. The suction tube includes multiple openings which may be used to evacuate secretions which pool above the inflatable cuff. In addition, the inflatable cuff includes a section immediately adjacent to the end of the suction tube that is less flexible than the rest of the inflatable cuff, to insure that the flexible material of the inflatable cuff is not sucked up against the suction tube openings. The endotracheal tube described in the Porter III patent has the disadvantages noted above, that the single lumen suction tube may exert suction on the tracheal mucosa and thereby cause damage to the mucosa. Further, the Porter III device is of a relatively complex design, requiring difficult processing, resulting in expensive production.

U.S. Pat. No. 5,143,062, issued to Peckham, discloses an endotracheal tube comprising a double lumen through which air may be circulated, creating an indirect gentle suction through a suction eye communicating with the distal ends of the lumens, and located at a position proximal to the inflation cuff. This design, however, does not provide adequate suction necessary for aspirating secretions and is easily occluded.

In fact, one problem that frequently arises in many of these catheters is that the suction port becomes occluded with secretions, rendering the function unusable. As such, what is needed is a multilumen catheter capable of suctioning secretions which have pooled above the inflatable cuff in a manner sufficient to accomplish the task but not so strong so as to cause damage to the mucosa. The suction function on such a device would be capable of being cleaned of accumulated secretions, preferably while in use. The instant invention addresses these problems by providing a multilumen tracheal tube and suction catheter system with a rinse function.

### SUMMARY OF THE INVENTION

The present invention improves upon a tracheal tube by incorporating a rinse lumen therein that enables suctioning of fluids, rinsing of secretions accumulated within the suction lumen while maintaining the rinse function and minimizing contact of the rinse liquid with the subglottic space. In one embodiment, the tracheal tube is formed from a flexible cannula having a length, a distal end, and a proximal end. The cannula consists of a plurality of walls extending substantially along the length of the cannula, dividing the cannula into a plurality of separate lumens including a respiratory lumen, a suction lumen, a rinse lumen, and an inflation lumen. An inflatable cuff surrounds the cannula proximal to the distal end. The inflatable cuff is adapted to seal the trachea of a patient. The inflation lumen is in fluid communication with the inflatable cuff. A closeable port extends through a side wall of the cannula proximal to the inflatable cuff. The port is in fluid communication with the suction lumen. The rinse lumen may terminate within the suction lumen proximal to the port or may terminate within a chamber formed within the suction lumen, the chamber being proximate to the port. A closure mechanism for selectively occluding the port and preventing fluid transfer from the suction lumen to the subglottic space is provided. The closure mechanism is actuated by an activator capable of selectively engaging and disengaging the closure mechanism.

In other embodiments, the closure mechanism may be in the form of a plug attached to a slidable member, the slidable member extending axially along the cannula and terminating at a user manipulable end, the user manipulable end comprising the activator.

Other embodiments may provide for a slidable member having a surface that is movable between at least two positions, the slidable member movable in response to liquid impingement upon the surface, the surface comprising the activator.

In still other embodiments, the tracheal tube may have an inflatable cuff having a shape to block a trachea beneath a glottis of the patient. A cannula may be disposed through the inflatable cuff. Such a cannula may contain a respiratory lumen, a suction lumen, and a rinse lumen. The suction lumen may have a port for suctioning a subglottic space external to the cannula while simultaneously enabling ventilation through the respiratory lumen. The rinse lumen may terminate within the suction lumen proximate to the port or may terminate within a chamber formed within the suction lumen, the chamber being proximate to the port.

In another embodiment a method of suctioning fluids from the subglottic space within an intubated patient is described. The method includes inserting a multilumen catheter into a patient's trachea, and inflating a cuff so as to sealingly engage the walls of the trachea to minimize the flow of fluids from the subglottic space into the patients lungs. The patient may be continuously ventilated through at least one lumen of the multilumen catheter. Suctioning of fluids from the subglottic space may be conducted through at least one other lumen. This lumen should have a port extending through a side wall of the catheter proximate to the cuff to access such fluids. This suction lumen may be rinsed by introducing a rinse liquid into the suction lumen proximate to the port through at least another lumen while suctioning fluids from the subglottic space. Rinsing may be accomplished under turbulent flow conditions, including as a spray. A closure mechanism for selectively occluding the port and preventing fluid transfer from the suction lumen to the subglottic space is provided. The method includes the steps of activating the closure mechanism so as to occlude the port, irrigating the suction lumen with the rinse liquid introduced into the suction lumen through the rinse lumen, maintaining suction on the suction lumen so as to evacuate the suction lumen of rinse liquid, all while maintaining ventilation to the patient.

Other objects, advantages and applications of the present invention will be made clear by the following detailed description of a preferred embodiment of the invention and the accompanying drawings wherein reference numerals refer to like or equivalent structures.

### BRIEF DESCRIPTION OF THE DRAWINGS

FIG. 1 is an elevational view of one embodiment of a multilumen catheter in accordance with the present invention;

FIG. 2 is a cross-sectional view of the FIG. 1 catheter at through line 2-2;

FIG. 3 is a cross-sectional view of the FIG. 1 catheter in position within a patient;

FIG. 4 is a cross-sectional view of the FIG. 1 catheter showing the closure mechanism taken longitudinally through the catheter at the port region; and

FIG. 5 is a cross-sectional view of yet another embodiment of the FIG. 1 catheter showing an alternative embodiment of the closure mechanism taken longitudinally through the catheter at the port region.

### DETAILED DESCRIPTION

Reference will now be made to the drawings in which the various elements of the present invention will be given numeral designations and in which the invention will be discussed so as to enable one skilled in the art to make and use the invention. It is to be understood that the following description is only exemplary of the principles of the present invention, and should not be viewed as narrowing the pending claims. Those skilled in the art will appreciate that aspects of the various embodiments discussed may be interchanged and modified without departing from the scope and spirit of the invention.

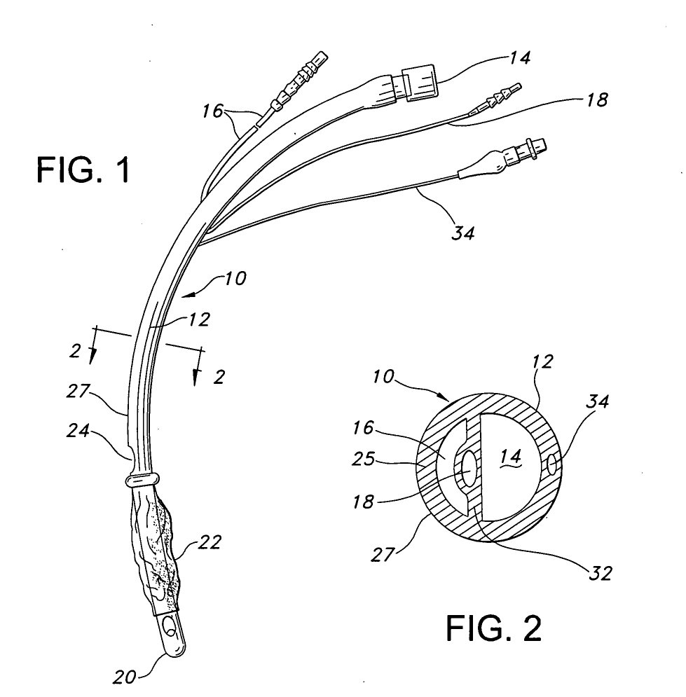

Referring to FIGS. 1 and 2, a tracheal tube 10 in accordance with one embodiment of the present invention is depicted. The tracheal tube 10 in the depicted embodiment is a multilumen cannula 12 having at least one respiratory lumen 14, at least one suction lumen 16, and at least one rinse lumen 18. In the embodiment, each of these lumens is at least partially internal to the cannula 12. The respiratory lumen 14 extends through the entire cannula 12 and is adapted to mechanically ventilate a patient (not shown). As such, a distal end 20 of the cannula 12 is situated within the upper respiratory system of the patient. A balloon, bladder, or inflatable cuff 22 is provided proximal to the distal end 20. The cuff 22 is shaped so that when inflated, it blocks the patient's trachea beneath the glottal area. This is known and understood by those skilled in the art to eliminate or at least to minimize the undesirable flow of fluids from the glottal and subglottal regions of the patient into the bronchus and lungs of the patient.

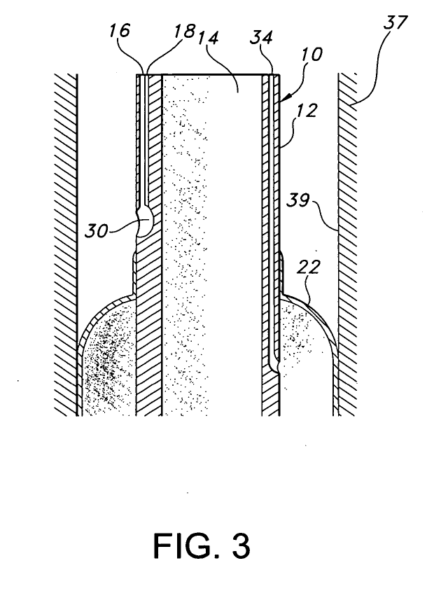

A port 24 extends from the suction lumen 16 through a wall 25 of the cannula 12 to an exterior surface 27 of the cannula 12. The port 24 in the depicted embodiment is proximate to an upper surface of the cuff 22. As such, the suction lumen 16 is adapted to suction fluids that collect above the cuff 22 in the patient's subglottic area without negatively impacting ventilation of the patient through the respiratory lumen 14. The rinse lumen 18, in this embodiment terminates within the cannula 12, specifically within the suction lumen 16 at an exit 30 as depicted in FIG. 3. Moreover, as depicted in FIG. 3, the rinse lumen 18 may terminate proximate to the port 24 which may be configured into the form of a chamber. It is to be understood that the rinse lumen 18 provides a path for the introduction of a rinse liquid 28. The rinse function is performed at the discretion of the caregiver in order to clean secretions and other liquids that may collect and potentially clog the suction lumen 16. In the embodiments depicted in each of FIGS. 1 through 5, the rinse lumen 18 is situated so as to contain the rinse liquid 28 within the suction lumen 16 and be suctioned along with the pooled liquids and other potentially clogging secretions contained within the suction lumen 16.

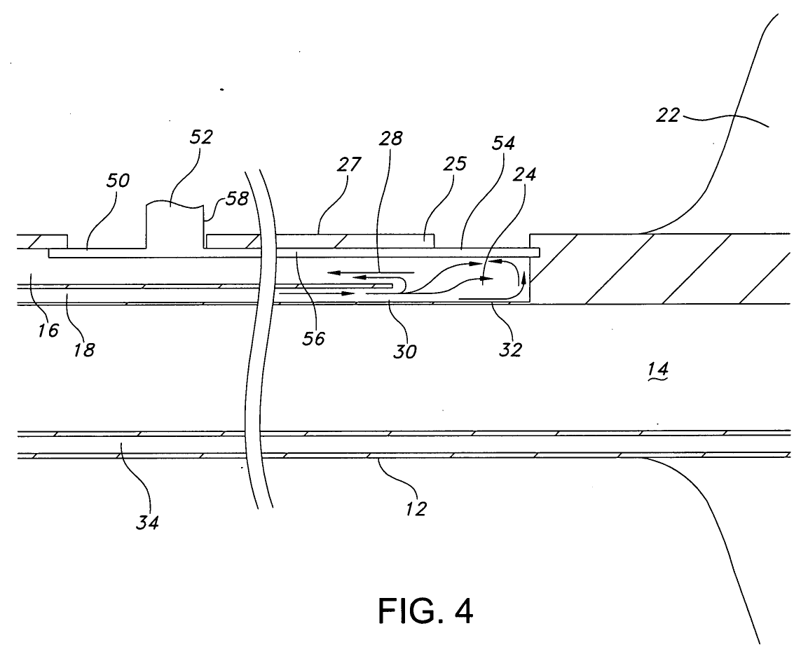

As shown in FIG. 4, to prevent the rinse liquid 28 from exiting the suction lumen 16 and entering the patient's subglottic space, a closure mechanism 50 is provided. The closure mechanism 50 is designed to occlude the port 24. An activator 52 may be provided to enable the caregiver to selectively engage and disengage the closure mechanism 50. In the FIG. 4 embodiment the closure mechanism 50 may comprise a plug 54 attached to a slidable member 56 which extends axially along the cannula 12 and terminates in a user manipulable end 58; the user manipulable end 58 comprising one embodiment of the activator 52. In other embodiments, the closure mechanism 50 may be moved by the liquid flow itself, thus fully automating the device based upon whether or not the rinse liquid 28 is introduced into the tracheal tube 10.

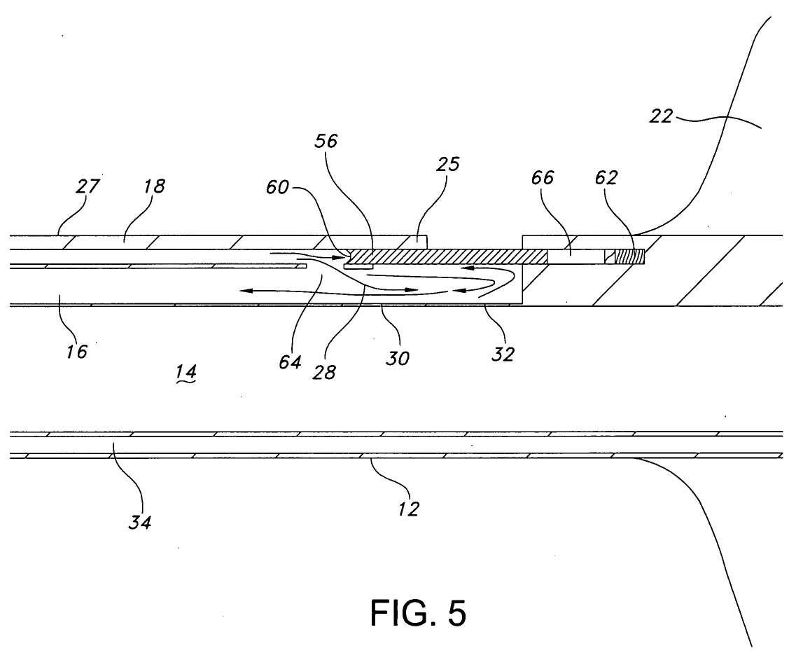

For example, in the FIG. 5 embodiment, the closure mechanism 50 may comprise the slidable member 56 that is movable between at least two positions, a first rinse lumen non-occluded position and a second rinse lumen occluded position. The slidable member 56 is movable in response to liquid impingement upon the surface 60, resulting in the slidable member 56 being forced by the rinse liquid 28 against a spring bias 62 until a passage 64 in the rinse lumen 18 is exposed allowing the rinse liquid 28 to exit the rinse lumen 18. Once the liquid impingement upon the surface 60 is removed, the slidable member 56 is caused to return to its initial position by the spring bias 62. A suitable passage 66 through the slidable member is provided so as not to occlude the port 24. Other embodiments are contemplated, in fact, one embodiment contemplates the use of a positive pressure differential between the suction lumen 16 and the subglottic space to cause the port 24 to seal and prevent liquid from entering the subglottic space.

In certain embodiments, the closure mechanism 50 may seat against an interior surface of the suction port 24 as shown in FIG. 4, however, it may also be configured so that it is on an exterior surface of the cannula 12. The former arrangement would work synergistically with the flow of rinse liquid causing the closure mechanism 50 to seat more efficiently. The closure mechanism 50 may take on many configurations; examples include but are not limited to an inflatable balloon, a plug, a cover, a valve, or a slidable member. In the case of an inflatable balloon, a dedicated lumen connected to an inflatable balloon would be provided. The balloon upon inflation would seal the port 24 enabling rinsing of the suction lumen in accordance with the above description.

The rinse liquid 28 itself, may comprise water, saline, as well as some other biocompatible liquid. A medicament, for example, an antiseptic or an antibiotic, or a treatment such as a surfactant may be added to the rinse liquid to obtain a desired effect on the patient, or to ease suctioning and/or cleaning of the suction lumen 16. Since the main purpose of the rinse liquid 28 is to rinse and/or clean the suction lumen 16, introducing the liquid into the suction lumen 16 in a turbulent manner will enable better cleaning of the suction lumen. As such the exit 30 of the rinse lumen 18 may be configured so as to foster turbulent flow or a spray pattern as depicted in FIGS. 4 and 5. Moreover the shape of the exit 30 or the chamber may contribute to such turbulence or provide a volume within which a spray may desirably be directed.

Looking back once again to the cross sectional view of FIG. 2, one possible configuration of the tracheal tube 10 is depicted, more specifically a potential lumen arrangement is depicted within the cannula 12. As can be seen, the respiratory lumen 14 is separated from the suction lumen 16 by an internal wall 32 that extends substantially along the entire length of the cannula 12. Formed into the internal wall 32 is the rinse lumen 18 which as described above terminates or exits at exit 30 within the suction lumen 16 or within the chamber 26, either being proximate to the port 24. This configuration is of course only meant to suggest one possible arrangement.

Other arrangements are included in the spirit and scope of the invention. For example, the layout of the lumens within the cannula 12 may be altered, moreover, the rinse lumen 18 may be formed in another wall, such as wall 25 of the cannula or it may be a self contained lumen not embedded within any one of the walls of the cannula 12. FIGS. 2-5 also depict an inflation lumen 34. The inflation lumen 34 is in fluid communication with the inflatable balloon or cuff 22 and as such controls inflation and deflation of the cuff 22 as would be understood by those of skill in the art. FIG. 3 depicts the tracheal tube 10 in position, that is, with the balloon 22 seated against the tracheal mucosa or tracheal wall 39 of the patient's trachea 37.

In use, the caregiver would insert the multilumen catheter or tracheal tube 10 into the patient's trachea 37 in a manner known and understood by those of skill in the art. The inflatable cuff 22 would be inflated through the inflation lumen 34 so as to sealingly engage the walls 39 of the patient's trachea 37. This would effectively prevent or at least minimize flow of undesirable fluids from the subglottic space into the bronchus and lungs. Ventilation of the patient through the respiratory lumen 14 may occur at this time and continue for as long as necessary.

At the discretion of the caregiver, the subglottic space within the patient's trachea may be suctioned through the suction lumen 16 via the port 24 through the wall 25 of the cannula 12. During suction, the suction lumen 16 may be rinsed by introduction of the rinse liquid 28 through the rinse lumen 18. The closure mechanism 50 is activated via the activator 52, which may be a function of the application of the rinse liquid 28 or may be via a user manipulable end 58 as described above. Actuation of the activator 52 occludes the port 28 and enables irrigation of the rinse lumen 18 without liquid 28 entering the subglottic space. Suctioning may continue thereby creating a flow of rinse liquid 28 from the rinse lumen 18 through the suction lumen 16 from where it is ejected and disposed. During this process, ventilation of the patient through the respiratory lumen 14 may of course continue unaffected.

Moreover, the rinse liquid 28 may be injected into the suction lumen 16 in a turbulent manner, including as a spray at or near the port 24 so as to better rinse the entire lumen 16. Rinsing the suction lumen 16 at the same time that suctioning is performed serves at least two functions, the first is that it minimizes the inadvertent flow of the rinse liquid 28 out of the cannula 12 and into the patient's subglottic space, and the second is that it increases the turbulent flow of the rinse liquid 28 at the port 24 and throughout the suction lumen 16 as well. Additionally, since the patient is isolated from the rinse liquid 28, higher pressures and greater suctioning may be used within the tracheal tube 10 without significantly increasing the risk to the patient that he will be subject to such pressures or vacuum.

As an alternative step, a treatment may be added to the rinse liquid 28 such as a medicament, for example, an antiseptic or an antibiotic. In that case, it may be desirable to allow the rinse liquid 28 to exit the cannula 12 and enter the subglottic space so as to gain the desired therapeutic effect prior to suctioning. Once the subglottic space is suctioned, the port 24 may be occluded as described above and rinse liquid 28 applied to the suction lumen 16.

As used herein and in the claims, the term “comprising” is inclusive or open-ended and does not exclude additional unrecited elements, compositional components, or method steps.

While various patents have been incorporated herein by reference, to the extent there is any inconsistency between incorporated material and that of the written specification, the written specification shall control. In addition, while the invention has been described in detail with respect to specific embodiments thereof, it will be apparent to those skilled in the art that various alterations, modifications and other changes may be made to the invention without departing from the spirit and scope of the present invention. It is therefore intended that the claims cover all such modifications, alterations and other changes encompassed by the appended claims.

We claim:

1. A tracheal tube comprising:
a cannula having a respiratory lumen, a suction lumen, and a rinse lumen, the suction lumen terminating in a port for suctioning a subglottic space external to the cannula while simultaneously enabling ventilation through the respiratory lumen, the rinse lumen terminating within the suction lumen proximate to the port;
a closure mechanism for selectively occluding the port and preventing fluid transfer from the suction lumen to the subglottic space; and
an activator for selectively engaging and disengaging the closure mechanism.

2. The tracheal tube of claim 1 wherein the closure mechanism comprises a plug attached to a slidable member, the slidable member extending axially along the cannula and terminating at a user manipulable end, the user manipulable end comprising the activator.

3. The tracheal tube of claim 1 wherein the closure mechanism comprises a slidable member having a surface that is movable between at least two positions, the slidable member movable in response to liquid impingement upon the surface, the surface comprising the activator.

4. The tracheal tube of claim 3 wherein introduction of a rinse liquid into the rinse lumen impinges upon the activator causing the slidable member to move from a first position to a second position, the first position characterized in that the slidable member is positioned away from the port, the second position characterized in that the slidable member occludes the port.

5. The tracheal tube of claim 1 wherein the closure mechanism seats against an interior surface of the suction port.

6. The tracheal tube of claim 1 wherein a positive pressure differential between the suction lumen and the subglottic space seals the port from liquid flow into the subglottic space.

7. The tracheal tube of claim 1 wherein the closure mechanism comprises an inflatable balloon for sealing against and occluding the port when inflated.

8. The tracheal tube of claim 1 wherein the closure mechanism comprises any of a plug, a cover, a valve, an inflatable balloon, alone or in combination.

9. The tracheal tube of claim 1 comprising a source of rinse liquid for introduction and passage through the rinse lumen.

10. A method of rinsing a suction lumen associated with a tracheal tube comprising:
intubating a patient with a tracheal tube, the tracheal tube having a distal end, a proximal end, an inflatable cuff proximate to the distal end sealing the trachea from the lungs at the subglottic region, and a respiratory lumen;
providing a suction lumen terminating in a port located proximal to the cuff for suctioning a subglottic space external to the cannula while simultaneously enabling ventilation through the respiratory lumen;
providing a rinse lumen terminating within the suction lumen proximate to the port;
providing a closure mechanism for selectively occluding the port and preventing fluid transfer from the suction lumen to the subglottic space;
activating the closure mechanism so as to occlude the port;
irrigating the suction lumen with a rinse liquid introduced into the suction lumen through a rinse lumen;
maintaining suction on the suction lumen so as to evacuate the suction lumen of rinse liquid; and
maintaining ventilation to the patient while irrigating and suctioning.

11. The method of claim 10 wherein the rinse liquid comprises a biocompatible liquid such as water or saline.

12. The method of claim 10 wherein the rinse liquid comprises an antiseptic.

13. The method of claim 10 wherein the rinse liquid is introduced into the suction lumen under turbulent flow conditions.

14. The method of claim 10 wherein the rinse liquid is introduced into the suction lumen as a spray.

15. A method of rinsing a suction lumen associated with a tracheal tube while simultaneously ventilating an intubated patient comprising:
intubating a patient with a tracheal tube having a distal end and an inflatable cuff proximate to the distal end sealing the subglottic space from the lungs at the trachea;
occluding a suction port contained within a suction lumen extending through the tracheal tube and terminating at the suction port proximal to the inflatable cuff;
irrigating the suction lumen with a rinse liquid introduced into the suction lumen proximal to the suction port via a rinse lumen;
maintaining ventilation to the patient; and
suctioning the liquid from the suction lumen while minimizing contact of the rinse liquid with the subglottic space.

16. The method of claim 15 comprising engaging a slidable member that extends axially along the cannula and terminates at a user manipulable end, the opposite end terminating in a plug adapted to occlude the suction port.

17. The method of claim 15 wherein introduction of the rinse liquid into the rinse lumen impinges upon an activator causing a plug to move from a first position to a second position, the first position characterized in that the plug is positioned away from the port, the second position characterized in that the plug occludes the port.
